(19) United States
(12) Patent Application Publication
(10) Pub. No.: US 2025/0040995 A1
(43) Pub. Date: Feb. 6, 2025

(54) UPDATING ENB TO CT REGISTRATION USING INTRA-OP CAMERA

(71) Applicant: Covidien LP, Mansfield, MA (US)

(72) Inventors: Ofer Barasofsky, Tel Mond (IL); Ariel Birenbaum, Raanana (IL); Guy Alexandroni, Yehud-Monosson (IL); Steven J. Levine, Framingham, MA (US); Paul M. Loschak, Somerville, MA (US); Nathan J. Knutson, Long Lake, MN (US); Jing Zhao, Superior, CO (US); Jared N. Farlow, Los Angeles, CA (US); Badr Elmaanaoui, Belmont, MA (US); John W. Komp, Dillon, CO (US); Matthew C. Blanco, Boston (UA); Scott E.M. Frushour, Boulder, CO (US); Daniel Ovadia, Ramat Gan (IL); Shaked Pessah, Maccabim-Reut (IL)

(73) Assignee: Covidien LP, Mansfield, MA (US)

(21) Appl. No.: 18/787,631

(22) Filed: Jul. 29, 2024

Related U.S. Application Data
(60) Provisional application No. 63/530,812, filed on Aug. 4, 2023.

(51) Int. Cl. A61B 34/20

(52) U.S. Cl. CPC A61B 34/20; A61B 2034/2051; A61B 2034/2055; A61B 2034/2065

(57) ABSTRACT

A system for performing a surgical procedure includes a catheter including a camera and an electromagnetic sensor and a workstation operably coupled to the catheter, the workstation including a memory storing instructions, which when executed cause a processor to receive pre-procedure images of a patient's anatomy, generate a 3D representation of the patient's anatomy, identify first anatomical landmarks within the generated 3D representation, identify a location of the EM sensor within a reference coordinate frame, receive real-time images from the camera, identify second anatomical landmarks within the received real-time images corresponding to the identified first anatomical landmarks, identify a location of the camera within the reference coordinate frame using the identified second anatomical landmarks corresponding to the identified first anatomical landmarks, and register a location of the catheter to the 3D representation using the identified locations of the EM sensor and the camera within the reference coordinate frame.

### BACKGROUND

### Technical Field

The present disclosure relates to the field of navigating medical devices within a patient, and in particular, planning a pathway though a luminal network of a patient and navigating medical devices to a target.

### Description of Related Art

There are several commonly applied medical methods, such as endoscopic procedures or minimally invasive procedures, for treating various maladies affecting organs including the liver, brain, heart, lungs, gall bladder, kidneys, and bones. Often, one or more imaging modalities, such as magnetic resonance imaging (MRI), ultrasound imaging, computed tomography (CT), cone-beam computed tomography (CBCT) or fluoroscopy (including 3D fluoroscopy) are employed by clinicians to identify and navigate to areas of interest within a patient and ultimately a target for biopsy or treatment. In some procedures, pre-operative scans may be utilized for target identification and intraoperative guidance. However, real-time imaging may be required to obtain a more accurate and current image of the target area. Furthermore, real-time image data displaying the current location of a medical device with respect to the target and its surroundings may be needed to navigate the medical device to the target in a safe and accurate manner (e.g., without causing damage to other organs or tissue).

For example, an endoscopic approach has proven useful in navigating to areas of interest within a patient. To enable the endoscopic approach endoscopic navigation systems have been developed that use previously acquired MRI data or CT image data to generate a three-dimensional (3D) rendering, model, or volume of the particular body part such as the lungs.

In some applications, the acquired MRI data or CT Image data may be acquired during the procedure (perioperatively). The resulting volume generated from the MRI scan or CT scan is then utilized to create a navigation plan to facilitate the advancement of the endoscope (or other suitable medical device) within the patient anatomy to an area of interest. In some cases, the volume generated may be used to update a previously created navigation plan. A locating or tracking system, such as an electromagnetic (EM) tracking system, or fiber-optic shape sensing system may be utilized in conjunction with, for example, CT data, to facilitate guidance of the endoscope to the area of interest.

However, CT-to-body divergence can cause inaccuracies in navigation using locating or tracking systems, leading to the use of fluoroscopic navigation to identify a current position of the medical device and correcting the location of the medical device in the 3D model. As can be appreciated, these inaccuracies can lead to increased surgical times to correct the real-time position of the medical device within the 3D models and the use of fluoroscopy leads to additional set-up time and radiation exposure.

### SUMMARY

A system for performing a surgical procedure includes a catheter, the catheter including a camera and an electromagnetic (EM) sensor, and a workstation operably coupled to the catheter, the workstation including a memory and a processor, the memory storing instructions, which when executed by the processor cause the processor to receive pre-procedure images of a patient's anatomy, generate a 3-dimensional (3D) representation of the patient's anatomy based on the received pre-procedure images, identify first anatomical landmarks within the generated 3D representation of the patient's anatomy, identify a location of the EM sensor of the catheter within a reference coordinate frame using the EM sensor, receive real-time images of the patient's anatomy from the camera of the catheter, identify second anatomical landmarks within the received real-time images corresponding to the identified anatomical landmarks within the generated 3D representation of the patient's anatomy, identify a location of the camera within the reference coordinate frame using the identified second anatomical landmarks within the real-time images corresponding to the identified first anatomical landmarks within the generated 3D representation of the patient's anatomy, and register a location of the catheter to the 3D representation of the patient's anatomy using the identified locations of the EM sensor and the camera within the reference coordinate frame.

In aspects, the system may include the instructions storing thereon further instructions, which when executed by the processor cause the processor to determine a distance between the camera and an identified anatomical landmark of the identified second anatomical landmarks within the received real-time images.

In certain aspects, the EM sensor of the catheter may be disposed on the catheter at a predetermined distance from the camera.

In other aspects, the system may include the memory storing thereon further instructions, which when executed by the processor cause the processor to determine a distance between the camera and the identified anatomical landmark of the identified second anatomical landmarks within the received real-time images using the predetermined distance between the EM sensor and the camera.

In certain aspects, the system may include an extended working channel (EWC), the EWC configured to selectively receive the catheter and permit the catheter to access a luminal network of the patient.

In aspects, the system may include the memory storing thereon further instructions, which when executed by the processor cause the processor to continuously receive real-time images of the patient's anatomy captured by the camera as the catheter is navigated through a luminal network of the patient.

In other aspects, the system may include the memory storing thereon further instructions, which when executed by the processor cause the processor to continuously identify second anatomical landmarks within the received real-time images corresponding to the identified first anatomical landmarks within the generated 3D representation of the patient's anatomy as the catheter is navigated through the luminal network of the patient.

In accordance with another aspect of the disclosure, a system for performing a surgical procedure includes a catheter, the catheter having a camera configured to capture images of a patient's anatomy, an extended working channel (EWC), the EWC configured to selectively receive the catheter and permit the catheter to access a luminal network of the patient, wherein the EWC includes an electromagnetic (EM) sensor, and a workstation operably coupled to the catheter, the workstation including a memory and a processor, the memory storing instructions, which when executed by the processor cause the processor to generate a 3-dimensional (3D) representation of the patient's anatomy based on pre-procedure images of the patient's anatomy, identify first anatomical landmarks within the generated 3D representation of the patient's anatomy, receive real-time images of the patient's anatomy from the camera of the catheter, identify second anatomical landmarks within the received real-time images corresponding to the identified first anatomical landmarks within the generated 3D representation of the patient's anatomy, identify a location of the catheter within the reference coordinate frame using the identified second anatomical landmarks within the real-time images corresponding to the identified first anatomical landmarks within the generated 3D representation of the patient's anatomy, and register a location of the catheter to the 3D representation of the patient's anatomy using the identified location of the catheter within the reference coordinate frame.

In aspects, the system may include the memory storing thereon further instructions, which when executed by the processor cause the processor to identify a location of the EM sensor of the EWC within the reference coordinate frame, wherein the location of the catheter is registered to the 3D representation of the patient's anatomy using both the identified location of the EM sensor and the identified location of the catheter.

In other aspects, the camera may be disposed a predetermined distance beyond the EM sensor of the EWC.

In certain aspects, the catheter may be configured to transition between a first, locked position where the catheter is inhibited from moving relative to the EWC and a second, unlocked position where the catheter is permitted to move relative to the EWC.

In other aspects, the system may include the memory storing thereon further instructions, which when executed by the processor cause the processor to determine a distance between the camera and an identified anatomical landmark of the identified second anatomical landmarks within the received real-time images using the predetermined distance between the EM sensor and the camera.

In aspects, the system may include the memory storing thereon further instructions, which when executed by the processor cause the processor to continuously receive real-time images of the patient's anatomy captured by the camera as the catheter is navigated through a luminal network of the patient.

In certain aspects, the system may include the memory storing thereon further instructions, which when executed by the processor cause the processor to continuously identify second anatomical landmarks within the received real-time images corresponding to the identified first anatomical landmarks within the generated 3D representation of the patient's anatomy as the catheter is navigated through the luminal network of the patient.

In accordance with another aspect of the disclosure, a method of registering a location of a medical device to a 3D representation of a patient's luminal network includes generating a 3-dimensional (3D) representation of a patient's luminal network based on pre-procedure images of the patient's anatomy, identifying first anatomical landmarks within the generated 3D representation of the patient's luminal network, identifying a plurality of locations of an electromagnetic (EM) sensor disposed on a catheter as the catheter is navigated through the luminal network of the patient, receiving a plurality of real-time images captured by a camera disposed on the catheter as the catheter is navigated through the luminal network of the patient, identifying second anatomical landmarks within the received real-time images corresponding to the identified first anatomical landmarks within the generated 3D representation of the patient's anatomy, identifying a location of the camera within the reference coordinate frame using the identified second anatomical landmarks within the real-time images corresponding to the identified first anatomical landmarks within the generated 3D representation of the patient's anatomy, and registering a location of the catheter to the 3D representation of the patient's anatomy using the identified positions of the EM sensor and the camera within the reference coordinate frame.

In aspects, the method may include determining a distance between the camera and an identified anatomical landmark of the identified second anatomical landmarks within the received real-time images, wherein the location of the camera within the reference coordinate frame is identified using the determined distance.

In certain aspects, the distance between the camera and the identified anatomical landmark of the identified second anatomical landmarks within the received real-time images may be determined using a pre-determined distance between the EM sensor and the camera.

In other aspects, the method may include advancing the catheter within an extended working channel (EWC) to gain access to the patient's luminal network.

In aspects, receiving a plurality of real-time images captured by the camera may include continuously receiving the plurality of real-time images from the camera as the catheter is navigated through the luminal network of the patient.

In other aspects, identifying second anatomical landmarks within the received real-time images may include continuously analyzing the continuously received plurality of real-time images to identify second anatomical landmarks within the received real-time images corresponding to the identified first anatomical landmarks within the generated 3D representation of the patient's anatomy.

### BRIEF DESCRIPTION OF THE DRAWINGS

Various aspects and embodiments of the disclosure are described hereinbelow with references to the drawings, wherein:

FIG. 1 is a schematic view of a surgical system provided in accordance with the present disclosure;

FIG. 2 is a schematic view of a workstation of the surgical system of FIG. 1;

FIG. 3 is a depiction of a graphical user interface of the surgical system of FIG. 1 illustrating a 3D representation of a patient's airways and location data of a catheter of the surgical system of FIG. 1;

FIG. 4 is a depiction of the graphical user interface of FIG. 3 illustrating an identified anatomical landmark within the 3D representation;

FIG. 5 is a depiction of the graphical user interface of FIG. 3 illustrating an identified anatomical landmark within a real-time image of the patient's airways captured by the catheter of the surgical system of FIG. 1;

FIG. 6A is a flow diagram of a method of registering a location of a medical device to a 3D model of a patient's luminal network;

FIG. 6B is a continuation of the flow diagram of FIG. 6A;

FIG. 7A is a flow diagram of another method of registering a location of a medical device to a 3D model of a patient's luminal network in accordance with the disclosure;

FIG. 7B is a continuation of the flow diagram of FIG. 7A;

FIG. 8 is a flow diagram of another method of registering a location of a medical device to a 3D model of a patient's luminal network in accordance with the disclosure;

FIG. 9 is a perspective view of a robotic surgical system of the surgical system of FIG. 1; and

FIG. 10 is an exploded view of a drive mechanism of an extended working channel of the surgical system of FIG. 1.

### DETAILED DESCRIPTION

This disclosure is directed to a surgical system configured to enable navigation of a medical device through a luminal network of a patient, such as for example the lungs. The surgical system includes a bronchoscope, through which an extended working channel (EWC), which may be a smart extended working channel (sEWC) including an electromagnetic (EM) sensor, is advanced to permit access of a catheter to the luminal network of the patient. As compared to an EWC, the sEWC includes an EM sensor disposed on or adjacent to a distal end of the sEWC that is configured for use with an electromagnetic navigation (EMN) or tracking system, which tracks the location of EM sensors, such as for example, the EM sensor of the sEWC. The catheter includes a camera disposed on or adjacent to a distal end of the catheter that is configured to capture real-time images of the patient's anatomy as the catheter is navigated through the luminal network of the patient. In this manner, the catheter is advanced through the sEWC and into the luminal network of the patient. It is envisioned that the catheter may be selectively locked to the sEWC to selectively inhibit, or permit, movement of the catheter relative to the sEWC. In embodiments, the catheter may include an EM sensor disposed on or adjacent to the distal end of the catheter.

The surgical system generates a 3-dimensional (3D) representation of the airways of the patient using pre-procedure images, such as for example, CT, CBCT, or MRI images and identifies anatomical landmarks within the 3D representation, such as for example, bifurcations or lesions. During a registration process of a surgical procedure, a location of the EM sensor of the sEWC is periodically identified and stored as a data point as the sEWC and catheter are navigated through the luminal network of the patient. As can be appreciated, the registration process may require the sEWC and catheter to be navigated within and survey particular portions of the patient's luminal network, such as for example, the right upper lobe, the left upper lobe, the right lower lobe, the left lower lobe, and the right middle lobe. As can be appreciated, the 3D representation generated from previously acquired images may not provide a basis sufficient for accurate registration or guidance of medical devices or tools to a target during a navigation phase of the surgical procedure. In some cases, the inaccuracy is caused by deformation of the patient's lungs during the surgical procedure relative to the lungs at the time of the acquisition of the previously acquired images. This is known as CT-to-body divergence. To mitigate CT-to-Body divergence and improve the accuracy of the registration process, the surgical system captures real-time images of the patient's anatomy from the camera disposed on the catheter as the catheter is navigated through the patient's luminal network. The real-time images captured by the camera are analyzed and the surgical system identifies anatomical features within the real-time images corresponding to the identified anatomical features within the 3D representation of the airways of the patient. The location of the camera when the image having the anatomical feature match is determined by determining a distance between the camera and the anatomical feature within the real-time image. This location of the camera, and therefore, the catheter, within the luminal network of the patient is stored as another data point. With the required portions of the airways of the patient surveyed, the stored data points, including the EM sensor locations and the camera locations, are used to generate a shape generally corresponding to the 3D representation of the airways of the patient. The shape is used to register the location of the EM sensor, and therefore, the sEWC, to the 3D representation of the airways of the patient.

It is envisioned that the surgical system may synthesize or otherwise generate virtual images from the 3D representation at various camera poses in proximity to the estimated location of the EM sensor within the airways of the patient. In this manner, a location within the 3D representation corresponding to the location data obtained from the EM sensors. The system generates virtual 2D or 3D images from the 3D representation corresponding to different perspectives or poses of the virtual camera viewing the patient's airways within the 3D representation. The real-time images captured by the camera are compared to the generated 2D or 3D virtual images and the virtual 2D or 3D image having a pose that most closely approximates the pose of the camera is identified. In this manner, the location of the identified virtual image within the 3D representation is correlated to the location of the EM sensors of the catheter within the reference coordinate frame and is recorded and utilized in addition to the location data obtained by the EM sensors to register a location of the catheter to the 3D representation.

Although generally described herein as utilizing a point cloud (e.g., for example, a plurality of location data points), it is envisioned that registration can be completed utilizing any number of location data points, and in one non-limiting embodiment, may utilize only a single location data point, such as for example, a known landmark within the airways of the patient. In this manner, the catheter is navigated to a location within the airways of the patient where a field of view of the camera captures a clear view of a main carina (e.g., for example, the tracheal carina). With a clear view of the main carina, an image of the main carina is obtained by the camera, which is analyzed to determine the pose of the camera 74 relative to the bronchial tree map of the 3D representation from which the captured image was obtained. As can be appreciated, the location of the EM sensors within the reference coordinate frame at the time the image of the main carina was captured is known, and using the determined pose of the camera the 3D representation can be registered to the reference coordinate frame.

With the initial registration of the 3D representation to the reference coordinate frame determined, it is envisioned that the initial registration of the 3D representation to the reference coordinate frame can be updated and/or refined by obtaining a plurality of location data points using the EM sensors as the catheter is advanced within the airways of the patient. In this manner, a point cloud is generated from the plurality of location data points. In embodiments, an algorithm, such as a machine learning algorithm, may be used to apply a greater weight to more recently obtained location data of the EM sensor during registration.

In embodiments, the system may utilize modalities other than virtual bronchoscopy, such as for example, generating a depth map (e.g., for example, a depth buffer) from the real-time images captured by the camera of the catheter. A depth map is estimated from the captured real-time image, which is converted into a 3D point cloud, and the resultant 3D point cloud is registered to the 3D representation

These and other aspects of the disclosure will be described in further detail hereinbelow. Although generally described with reference to the lung, it is contemplated that the systems and methods described herein may be used with any structure within the patient's body, such as the liver, kidney, prostate, gynecological, amongst others.

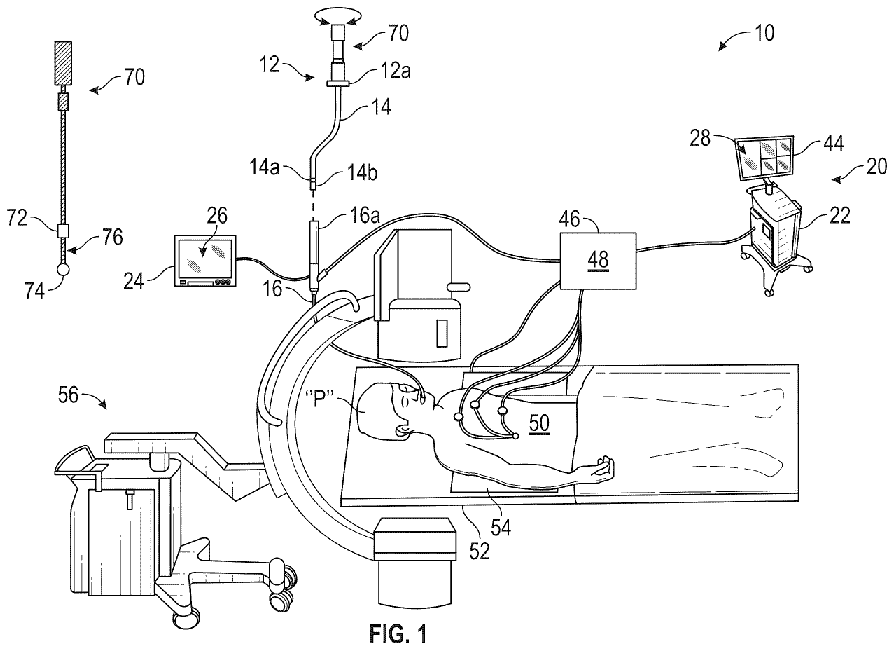

Turning now to the drawings, FIG. 1 illustrates a system 10 in accordance with the disclosure facilitating navigation of a medical device through a luminal network and to an area of interest. As will be described in further detail hereinbelow, the surgical system 10 is generally configured to identify target tissue, automatically register real-time images captured by a surgical instrument to a generated 3-dimensional (3D) model, and navigate the surgical instrument to the target tissue.

The system 10 includes a catheter guide assembly 12 including an extended working channel (EWC) 14, which may be a smart extended working channel (sEWC) including an electromagnetic (EM) sensor. In one embodiment, the sEWC 14 is inserted into a bronchoscope 16 for access to a luminal network of the patient P. In this manner, the sEWC 14 may be inserted into a working channel of the bronchoscope 16 for navigation through a patient's luminal network, such as for example, the lungs. It is envisioned that the sEWC 14 may itself include imaging capabilities via an integrated camera or optics component (not shown) and therefore, a separate bronchoscope 16 is not strictly required. In embodiments, the sEWC 14 may be selectively locked to the bronchoscope 16 using a bronchoscope adapter 16a. In this manner, the bronchoscope adapter 16a is configured to permit motion of the sEWC 14 relative to the bronchoscope 16 (which may be referred to as an unlocked state of the bronchoscope adapter 16a) or inhibit motion of the sEWC 14 relative to the bronchoscope 16 (which may be referred to as a locked state of the bronchoscope adapter 16a). Bronchoscope adapters 16a are currently marketed and sold by Medtronic PLC under the brand names EDGE® Bronchoscope Adapter or the ILLUMISITE® Bronchoscope Adapter, and are contemplated as being usable with the disclosure.

As compared to an EWC, the sEWC 14 may include one or more EM sensors 14a disposed in or on the sEWC 14 at a predetermined distance from the distal end 14b of the sEWC 14. It is contemplated that the EM sensor 14a may be a five degree-of-freedom sensor or a six degree-of-freedom sensor. As can be appreciated, the position and orientation of the EM sensor 14a of the sEWC relative to a reference coordinate system, and thus a distal portion of the sEWC 14 within an electromagnetic field can be derived. Catheter guide assemblies 12 are currently marketed and sold by Medtronic PLC under the brand names SUPERDIMENSION® Procedure Kits, ILLUMISITE™ Endobronchial Procedure Kit, ILLUMISITE™ Navigation Catheters, or EDGE® Procedure Kits, and are contemplated as being usable with the disclosure.

A catheter 70, including one or more EM sensors 72, is inserted into the sEWC and selectively locked into position relative to the sEWC 14 such that the sensor 72 extends a predetermined distance beyond a distal tip of the sEWC 14. As can be appreciated, the EM sensor 72 disposed on the catheter 70 is separate from the EM sensor 14a disposed on the sEWC. The EM sensor 72 is disposed on or in the catheter 70 a predetermined distance from a distal end 76 of the catheter 70. In this manner, the system 10 is able to determine a position of a distal portion of the catheter 70 within the luminal network of the patient P. It is envisioned that the catheter 70 may be selectively locked relative to the sEWC 14 at any time, regardless of the position of the distal end 76 of the catheter 70 relative to the sEWC 14. It is contemplated that the catheter 70 may be selectively locked to a handle 12a of the catheter guide assembly 12 using any suitable means, such as for example, a snap fit, a press fit, a friction fit, a cam, one or more detents, threadable engagement, or a chuck clamp. It is envisioned that the EM sensor 72 may be a five degree-of-freedom sensor or a six degree-of-freedom sensor. As will be described in further detail hereinbelow, the position and orientation of the EM sensor 72 of the catheter 70 relative to a reference coordinate system, and thus a distal portion of the catheter 70, within an electromagnetic field can be derived.

At least one camera 74 is disposed on or adjacent the distal end 76 of the catheter 70 and is configured to capture, for example, still images, real-time images, or real-time video. Although generally described as being disposed on the distal end 76 of the catheter 70, it is envisioned that the camera 74 may be disposed on any suitable location on the camera 70, such as for example, a sidewall. In embodiments, the catheter 70 may include one or more light sources (not shown) disposed on or adjacent to the distal end 76 of the catheter 70 or any other suitable location (e.g., for example, a side surface or a protuberance). The light source may be or may include, for example, a light emitting diode (LED), an optical fiber connected to a light source that is located external to the patient, or combinations thereof, and may emit one or more of white, IR, or near infrared (NIR) light. In this manner, the camera 74 may be, for example, a white light camera, IR camera, or NIR camera, a camera that is capable of capturing white light and NIR light, or combinations thereof. In one non-limiting embodiment, the camera 74 is a white light mini complementary metal-oxide semiconductor (CMOS) camera, although it is contemplated that the camera 74 may be any suitable camera, such as for example, a charge-coupled device (CCD), a complementary metal-oxide-semiconductor (CMOS), a N-type metal-oxide-semiconductor (NMOS), and in embodiments, may be an infrared (IR) camera, depending upon the design needs of the system 10. As can be appreciated, the camera 74 captures images of the patient's anatomy from a perspective of looking out from the distal end 76 of the catheter 70. In embodiments, the camera 74 may be a dual lens camera or a Red Blue Green and Depth (RGB-D) camera configured to identify a distance between the camera 74 and anatomical features within the patient's anatomy without departing from the scope of the disclosure. As described hereinabove, it is envisioned that the camera 74 may be disposed on the catheter 70, the sEWC 14, or the bronchoscope 16.

With continued reference to FIG. 1, the system 10 generally includes an operating table 52 configured to support a patient P and monitoring equipment 24 coupled to the sEWC 14, the bronchoscope 16, or the endoscope 70 (e.g., for example, a video display for displaying the video images received from the video imaging system of the bronchoscope 12 or the camera 74 of the catheter 70), a locating or tracking system 46 including a tracking module 48, a plurality of reference sensors 50 and a transmitter mat 54 including a plurality of incorporated markers, and a workstation 20 having a computing device 22 including software and/or hardware used to facilitate identification of a target, pathway planning to the target, navigation of a medical device to the target, and/or confirmation and or determination of placement of, for example, the sEWC 14, the bronchoscope 16, the catheter 70, or a surgical tool, relative to the target.

The tracking system 46 is, for example, a six degrees-of-freedom electromagnetic locating or tracking system, or other suitable system for determining position and orientation of, for example, a distal portion the sEWC 14, the bronchoscope 16, the catheter 70, or a surgical tool, for performing registration of a detected position of one or more of the EM sensors 14a or 72 and a three-dimensional (3D) model generated from a CT, CBCT, or MRI image scan. The tracking system 46 is configured for use with the sEWC 14 and the catheter 70, and particularly with the EM sensors 14a and 72.

Continuing with FIG. 1, the transmitter mat 54 is positioned beneath the patient P. The transmitter mat 54 generates an electromagnetic field around at least a portion of the patient P within which the position of the plurality of reference sensors 50 and the EM sensors 14a and 74 can be determined with the use of the tracking module 48. In one non-limiting embodiment, the transmitter mat 54 generates three or more electromagnetic fields. One or more of the reference sensors 50 are attached to the chest of the patient P. In embodiments, coordinates of the reference sensors 50 within the electromagnetic field generated by the transmitter mat 54 are sent to the computing device 22 where they are used to calculate a patient coordinate frame of reference (e.g., for example, a reference coordinate frame). As will be described in further detail hereinbelow, registration is generally performed using coordinate locations of the 3D model and 2D images from the planning phase, with the patient P's airways as observed through the bronchoscope 12 or catheter 70 and allow for the navigation phase to be undertaken with knowledge of the location of the EM sensors 14a and 72. It is envisioned that any one of the EM sensors 14a and 72 may be a single coil sensor that enables the system 10 to identify the position of the sEWC 14 or the catheter 70 within the EM field generated by the transmitter mat 54, although it is contemplated that the EM sensors 14a and 72 may be any suitable sensor and may be a sensor capable of enabling the system 10 to identify the position, orientation, and/or pose of the sEWC 14 or the catheter 70 within the EM field.

Although generally described with respect to EMN systems using EM sensors, the instant disclosure is not so limited and may be used in conjunction with flexible sensors, such as for example, fiber-bragg grating sensors, inertial measurement units (IMU), ultrasonic sensors, optical sensors, pose sensors (e.g., for example, ultra-wide band, global positioning system, fiber-bragg, radio-opaque markers), without sensors, or combinations thereof. It is contemplated that the devices and systems described herein may be used in conjunction with robotic systems such that robotic actuators drive the sEWC 14 or bronchoscope 16 proximate the target.

In accordance with aspects of the disclosure, the visualization of intra-body navigation of a medical device (e.g., for example a biopsy tool or a therapy tool), towards a target (e.g., for example, a lesion) may be a portion of a larger workflow of a navigation system. An imaging device 56 (e.g., for example, a CT imaging device, such as for example, a cone-beam computed tomography (CBCT) device, including but not limited to Medtronic PLC's O-arm™ system) capable of acquiring 2D and 3D images or video of the patient P is also included in the particular aspect of system 10. The images, sequence of images, or video captured by the imaging device 56 may be stored within the imaging device 56 or transmitted to the computing device 22 for storage, processing, and display. In embodiments, the imaging device 56 may move relative to the patient P so that images may be acquired from different angles or perspectives relative to the patient P to create a sequence of images, such as for example, a fluoroscopic video. The pose of the imaging device 56 relative to the patient P while capturing the images may be estimated via markers incorporated with the transmitter mat 54. The markers are positioned under the patient P, between the patient P and the operating table 52, and between the patient P and a radiation source or a sensing unit of the imaging device 56. The markers incorporated with the transmitter mat 54 may be two separate elements which may be coupled in a fixed manner or alternatively may be manufactured as a single unit. It is contemplated that the imaging device 56 may include a single imaging device or more than one imaging device.

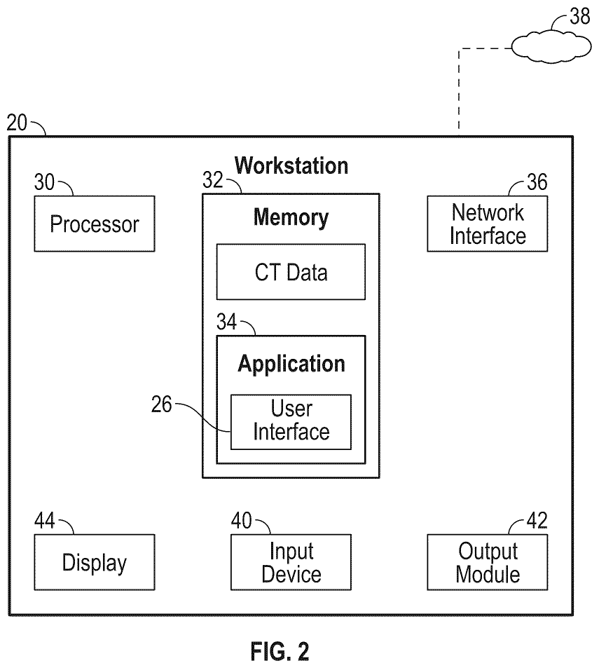

Continuing with FIG. 1 and with additional reference to FIG. 2, the workstation 20 includes a computer 22 and a display 24 that is configured to display one or more user interfaces 26 and/or 28. The workstation 20 may be a desktop computer or a tower configuration with the display 24 or may be a laptop computer or other computing device. The workstation 20 includes a processor 30 which executes software stored in a memory 32. The memory 32 may store video or other imaging data captured by the bronchoscope 16 or catheter 70 or pre-procedure images from, for example, a computer-tomography (CT) scan, Positron Emission Tomography (PET), Magnetic Resonance Imaging (MRI), Cone-beam CT, amongst others. In addition, the memory 32 may store one or more software applications 34 to be executed on the processor 30. Though not explicitly illustrated, the display 24 may be incorporated into a head mounted display such as an augmented reality (AR) headset such as the HoloLens offered by Microsoft Corp.

A network interface 36 enables the workstation 20 to communicate with a variety of other devices and systems via the Internet. The network interface 36 may connect the workstation 20 to the Internet via a wired or wireless connection. Additionally, or alternatively, the communication may be via an ad-hoc Bluetooth® or wireless network enabling communication with a wide-area network (WAN) and/or a local area network (LAN). The network interface 36 may connect to the Internet via one or more gateways, routers, and network address translation (NAT) devices. The network interface 36 may communicate with a cloud storage system 38, in which further image data and videos may be stored. The cloud storage system 38 may be remote from or on the premises of the hospital such as in a control or hospital information technology room. An input module 40 receives inputs from an input device such as a keyboard, a mouse, voice commands, amongst others. An output module 42 connects the processor 30 and the memory 32 to a variety of output devices such as the display 24. In embodiments, the workstation 20 may include its own display 44, which may be a touchscreen display.

In a planning or pre-procedure phase, the software application utilizes pre-procedure CT image data, either stored in the memory 32 or retrieved via the network interface 36, for generating and viewing a 3D model of the patient's anatomy, enabling the identification of target tissue TT on the 3D model (automatically, semi-automatically, or manually), and in embodiments, allowing for the selection of a pathway through the patient's anatomy to the target tissue. Examples of such an application is the ILOGIC® planning and navigation suites and the ILLUMISITE® planning and navigation suites currently marketed by Medtronic PLC. The 3D model may be displayed on the display 24 or another suitable display associated with the workstation 20, such as for example, the display 44, or in any other suitable fashion. Using the workstation 20, various views of the 3D model may be provided and/or the 3D model may be manipulated to facilitate identification of target tissue on the 3D model and/or selection of a suitable pathway to the target tissue.

It is envisioned that the 3D model may be generated by segmenting and reconstructing the airways of the patient P's lungs to generate a 3D airway tree 80. The reconstructed 3D airway tree includes various branches and bifurcations which, in embodiments, may be labeled using, for example, well accepted nomenclature such as RB1 (right branch 1), LB1 (left branch 1, or B1 (bifurcation one). In embodiments, the segmentation and labeling of the airways of the patient's lungs is performed to a resolution that includes terminal bronchioles having a diameter of approximately less than 1 mm. As can be appreciated, segmenting the airways of the patient P's lungs to terminal bronchioles improves the accuracy registration between the position of the sEWC 14 and catheter 70 and the 3D model, improves the accuracy of the pathway to the target, and improves the ability of the software application to identify the location of the sEWC 14 and catheter 70 within the airways and navigate the sEWC 14 and catheter 70 to the target tissue. Those of skill in the art will recognize that a variety of different algorithms may be employed to segment the CT image data set, including, for example, connected component, region growing, thresholding, clustering, watershed segmentation, or edge detection. It is envisioned that the entire reconstructed 3D airway tree may be labeled, or only branches or branch points within the reconstructed 3D airway tree that are located adjacent to the pathway to the target tissue.

In embodiments, the software stored in the memory 32 may identify and segment out a targeted critical structure within the 3D model. It is envisioned that the segmentation process may be performed automatically, manually, or a combination of both. The segmentation process isolates the targeted critical structure from the surrounding tissue in the 3D model and identifies its position within the 3D model. In embodiments, the software application segments the CT images to terminal bronchioles that are less than 1 mm in diameter such that branches and/or bifurcations are identified and labeled deep into the patient's luminal network. As an be appreciated, this position can be updated depending upon the view selected on the display 24 such that the view of the segmented targeted critical structure may approximate a view captured by the camera 74 of the catheter 70.

As can be appreciated, the 3D model generated from previously acquired images may not provide a basis sufficient for accurate registration or guidance of medical devices or tools to a target during a navigation phase of the surgical procedure. In some cases, the inaccuracy is caused by deformation of the patient's lungs during the surgical procedure relative to the lungs at the time of the acquisition of the previously acquired images. This deformation (CT-to-Body divergence) may be caused by many different factors including, for example, changes in the patient P's body when transitioning from between a sedated state and a non-sedated state, the bronchoscope 16, the sEWC 14, or the catheter 70 changing the patient P's pose, the bronchoscope 16, the sEWC 14, or the catheter 70 pushing the tissue, different lung volumes (e.g., for example, the previously acquired images are acquired during an inhale while navigation is performed as the patient P is breathing), different beds, a time period between when the previous images were captured and when the surgical procedure is being performed, a change in the lung shape due to, for example, a change in temperature or time of day between when the previous images were captured and when the surgical procedure is being performed, the effects of gravity on the patient P's lungs due to the length of time the patient P is laying on the operating table 52, or diseases that were not present or have progressed since the time when the previous images were captured.

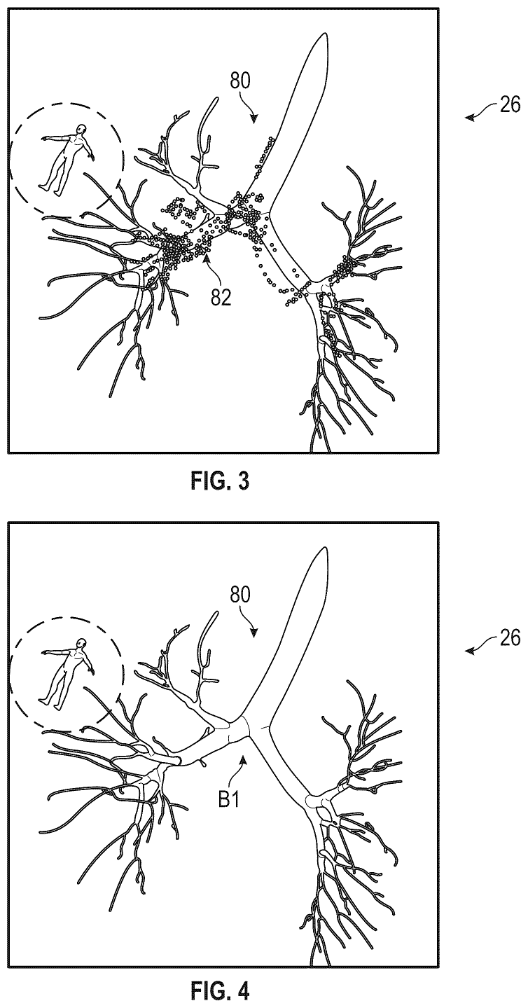

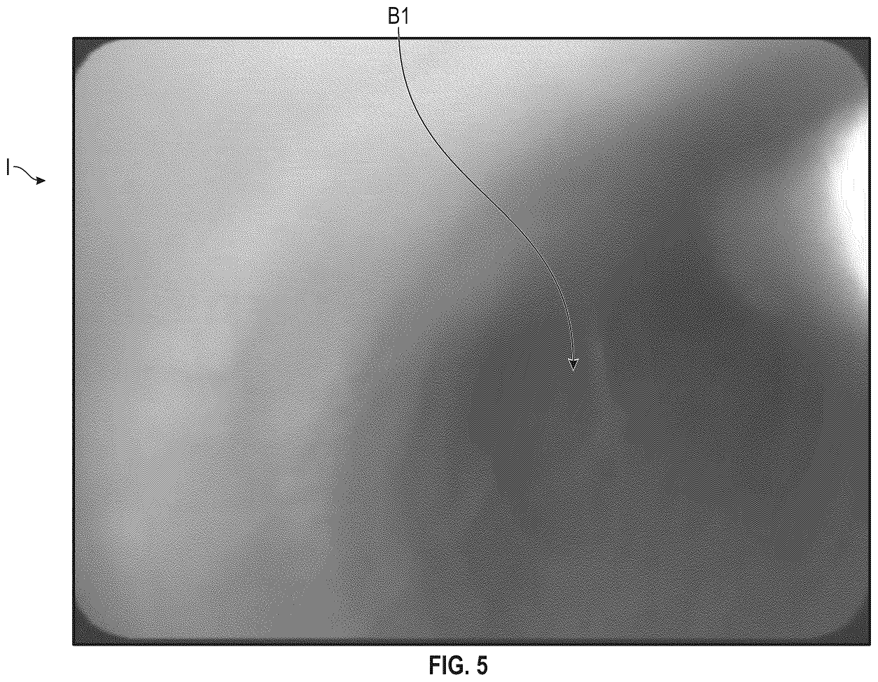

With additional reference to FIGS. 3 and 4, registration of the patient P's location on the transmitter mat 54 may be performed by moving the EM sensors 14a and/or 72 through the airways of the patient P. In this manner, the software stored on the memory 32 periodically determines the location of the EM sensors 14a or 72 within the coordinate system as the sEWC 14 of the catheter 70 is moving through the airways using the transmitter mat 54, the reference sensors 50, and the tracking system 46. The location data may be represented on the user interface 26 as a marker or other suitable visual indicator 82, a plurality of which develop a point cloud having a shape that may approximate the interior geometry of the 3D model 80. The shape resulting from this location data is compared to an interior geometry of passages of a 3D model 80, and a location correlation between the shape and the 3D model 80 based on the comparison is determined. In addition, the software identifies non-tissue space (e.g., for example, air filled cavities) in the 3D model. The software aligns, or registers, an image representing a location of the EM sensors 14a or 72 with the 3D model 80 and/or 2D images generated from the 3D model 80, which are based on the recorded location data and an assumption that the sEWC 14 or the catheter 70 remains located in non-tissue space in a patient's airways. In embodiments, a manual registration technique may be employed by navigating the sEWC 14 or catheter 70 with the EM sensors 14a and 72 to pre-specified locations in the lungs of the patient P, and manually correlating the images from the bronchoscope 16 or the catheter 70 to the model data of the 3D model. Although generally described herein as utilizing a point cloud (e.g., for example, a plurality of location data points), it is envisioned that registration can be completed utilizing any number of location data points, and in one non-limiting embodiment, may utilize only a single location data point.

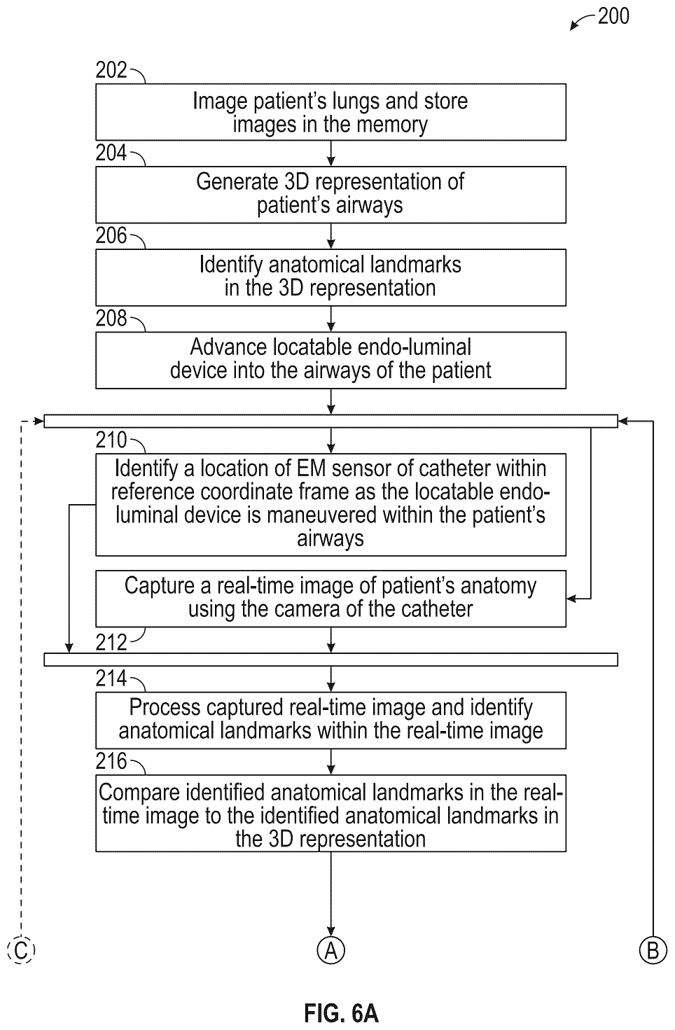

During registration, CT-to-Body divergence is mitigated by integrating real-time images captured by the camera 74 of the catheter 70 as the catheter 70 is moving through the patient P's airways. In this manner, the software stored on the memory 32 analyzes the pre-segmented pre-procedure CT model and identifies locations of anatomical landmarks, such as for example, bifurcations, airway walls, or lesions. In one non-limiting embodiment, the identified anatomical landmark is a bifurcation, labeled as B1 in the user interface 26 (FIG. 4). As the catheter 70 is moving through the airways, images “I” of the patient P's anatomy are captured using the camera 74 in real-time and are continuously segmented via the software stored in the memory 32 to identify anatomical landmarks within the real-time images I. The software stored on the memory 32 continuously analyzes the captured images I in real-time and identifies commonalities between the anatomical landmarks identified by the software application in the real-time images I and the pre-procedure images, illustrated as bifurcation B1 in FIG. 5. A distance between the camera 74 and the anatomical landmarks identified in the real-time images I is determined using, for example, the EM sensors 14a or 72, the predetermined distance between the EM sensors 14a or 72, and a known zoom level of the real-time images I captured by the camera 74, although it is contemplated that the distance between the camera 74 and the identified anatomical landmarks may be determined using any suitable means without departing from the scope of the present disclosure, such as for example, data obtained from a dual lens camera or RGB-D camera. Using the distance between the camera 74 and the anatomical landmark, the location of the sEWC 14 and/or the catheter 70 within the coordinate system is recorded and utilized in addition to the location data obtained by the EM sensors 14a and/or 72 to register a location of the sEWC 14 or the catheter 72 to the 3D model 80. In embodiments where a dual lens camera or RGB-D camera is utilized, a determined distance between the camera 74 and the identified anatomical landmark is compared to the location data obtained by the EM sensors 14a and/or 72. In this manner, a second distance between the camera 74 and the identified anatomical landmark is determined, adding redundancy and increasing the accuracy of the distance determination. It is contemplated that data points where distance determined using the camera 74 correlates to the distance determined using the EM sensors 14a and/or 72 may be weighted or otherwise afforded greater importance during registration.

Although generally described hereinabove as utilizing anatomical landmarks during the registration process, this disclosure is not so limited. In embodiments, the software stored on the memory 32 synthesizes or otherwise generates virtual images from the 3D model 80 at various virtual camera poses in proximity to the estimated location of the EM sensors 14a and/or 72 within the airways of the patient. In this manner, the software stored on the memory 32 identifies a location within the 3D model 80 corresponding to the location data obtained from the EM sensors 14a and/or 72. The software stored on the memory 32 generates virtual 2D or 3D images from the 3D model 80 corresponding to different perspectives or poses of the virtual camera viewing the patient's airways within the 3D model 80. The software stored on the memory 32 compares the real-time images I captured by the camera 74 of the catheter 70 to the generated virtual 2D or 3D images and identifies a generated virtual 2D or 3D image having a pose that most closely approximates the pose of the camera 74 of the catheter 70. In this manner, the location of the identified virtual image within the 3D model 80 is correlated to the location of the sEWC 14 and/or the catheter 70 within the coordinate system and is recorded and utilized in addition to the location data obtained by the EM sensors 14a and/or 72 to register a location of the sEWC 14 or the catheter 72 to the 3D model 80.

Although generally described herein as utilizing a point cloud (e.g., for example, a plurality of location data points), it is envisioned that registration can be completed utilizing any number of location data points, and in one non-limiting embodiment, may utilize only a single location data point, such as for example, a known landmark within the airways of the patient. In this manner, the catheter 70 is navigated to a location within the airways of the patient where a field of view of the camera 74 captures a clear view of a main carina (e.g., for example, the tracheal carina). With a clear view of the main carina, an image of the main carina is obtained by the camera 74 of the catheter 70. The software stored on the memory 32 analyzes the captured image of the main carina and determines the pose of the camera 74 relative to the bronchial tree map of the 3D model 80 from which the captured image was obtained. As can be appreciated, the location of the EM sensors 14a and/or 72 within the reference coordinate frame at the time the image of the main carina was captured is known, and using the determined pose of the camera 74, the 3D model 80 can be registered to the reference coordinate frame.

With the initial registration of the 3D model 80 to the reference coordinate frame determined, it is envisioned that the initial registration of the 3D model 80 to the reference coordinate frame can be updated and/or refined by obtaining a plurality of location data points using the EM sensors 14a and/or 72 as the catheter 70 is advanced within the airways of the patient and generating a point cloud as described in further detailed hereinabove. In embodiments, the software stored on the memory 32 may utilize an algorithm, such as a machine learning algorithm, to apply a greater weight to more recently obtained location data of the EM sensor 14a and/or 72 during registration.

In embodiments, the software stored on the memory 32 may utilize modalities other than virtual bronchoscopy, such as for example, generating a depth map (e.g., for example, a depth buffer) from the real-time images captured by the camera 74 of the catheter 70. The software stored on the memory 32 estimates the depth map from the real-time images and converts the estimated depth map into a 3D-point cloud. The resultant 3D-point cloud is registered to the 3D model 80 using the software stored on the memory 32. Those having skill in the art will recognize that various different transformations may be utilized to register the location of the sEWC 14 and/or the catheter 70 to the 3D model 80 without departing from the scope of the present disclosure.

As can be appreciated, these additional data points refine or otherwise improve the accuracy of the generated shape and therefore, improves the accuracy of registration by mitigating CT-to-Body divergence. Although generally described as being utilized for a global registration process, it is envisioned that registration between the location of the EM sensors 14a and/or 72 may be performed locally, such as for example, adjacent to an area or interest or target using the camera 74 of the catheter 70 as described hereinabove without departing from the scope of the present disclosure. As can be appreciated, integrating the real-time images I captured by the camera 74 into the registration process minimizes the need to utilize fluoroscopy, CBCT, or other modalities to identify the position of the endoscope within the patient P's airways.

Although generally described as utilizing pre-procedure images, it is envisioned that the identified branches or bifurcations may be continuously updated based on intraprocedural images captured perioperatively. As can be appreciated, by updating the images utilized to identify the branches or bifurcations and the labeling thereof, the 3D model 80 can more accurately reflect the real time condition of the lungs, taking into account, for example, atelectasis or mechanical deformation of the airways. Although generally described with respect to the airways of a patient's lungs, it is envisioned that the software stored in the memory 32 may identify and/or label portions of the bronchial and/or pulmonary circulatory system within the lung. These labels may appear concurrently with the labels of branches or bifurcations of the airways displayed to the user.

In accordance with aspects of the disclosure, the visualization of intra-body navigation of a medical device (e.g., for example a biopsy tool or a therapy tool), towards a target (e.g., for example, a lesion) may be a portion of a larger workflow of a navigation system. An imaging device 56 (e.g., for example, a CT imaging device, such as for example, a cone-beam computed tomography (CBCT) device, including but not limited to Medtronic plc's O-arm™ system) capable of acquiring 2D and 3D images or video of the patient P is also included in the particular aspect of system 10. The images, sequence of images, or video captured by the imaging device 56 may be stored within the imaging device 56 or transmitted to the computing device 22 for storage, processing, and display. In embodiments, the imaging device 56 may move relative to the patient P so that images may be acquired from different angles or perspectives relative to the patient P to create a sequence of images, such as for example, a fluoroscopic video. The pose of the imaging device 56 relative to the patient P while capturing the images may be estimated via markers incorporated with the transmitter mat 54. The markers are positioned under the patient P, between the patient P and the operating table 52, and between the patient P and a radiation source or a sensing unit of the imaging device 56. The markers incorporated with the transmitter mat 54 may be two separate elements which may be coupled in a fixed manner or alternatively may be manufactured as a single unit. It is contemplated that the imaging device 56 may include a single imaging device or more than one imaging device.

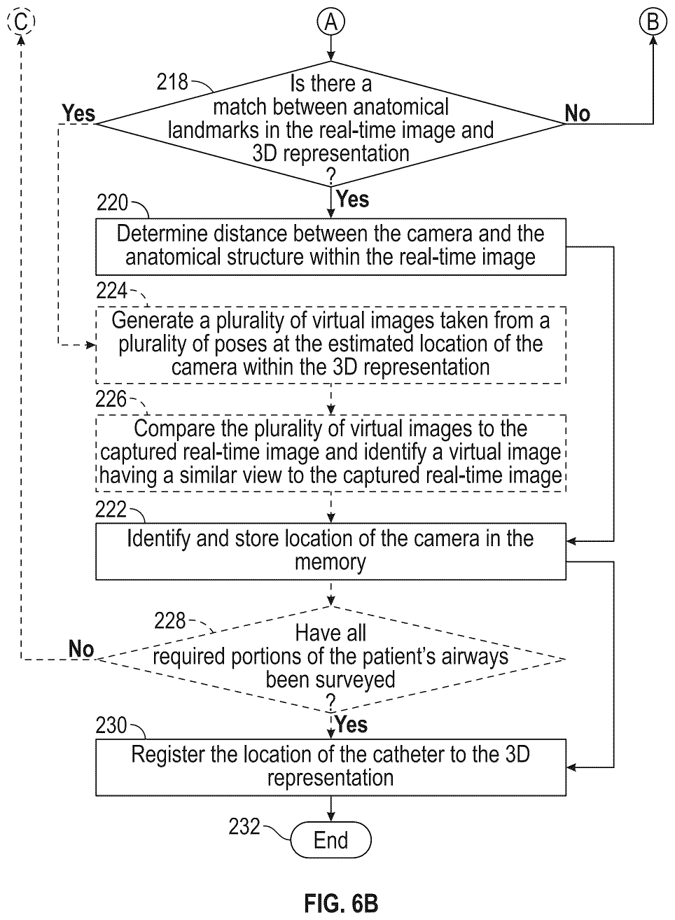

With reference to FIGS. 6A and 6B, a method of registering a location of a medical device to a 3D model of a patient's luminal network is described and generally identified by reference numeral 200. Initially, in step 202, the patient's lungs are imaged using any suitable imaging modality (e.g., for example, CT, MRI, or CBCT) and the images are stored on the memory 32 associated with the workstation 20. As can be appreciated, imaging of the patient's lungs of step 202 may be done at any suitable time, such as for example, pre-operatively, intra-operatively, or peri-operatively. In step 204, the images stored on the memory 32 are utilized to generate and view a 3D representation of the airways of the patient P's lungs. Thereafter, relevant anatomical landmarks or features, such as for example, a bifurcation, are identified within the 3D representation in step 206. In some aspects, step 206 or a step between steps 206 and 208 may include creating a plan including a pathway to a target in the 3D representation. The steps before step 208 may be performed preoperatively.

Once the 3D representation of the airways is generated and anatomical landmarks identified within the 3D representation, a locatable endo-luminal device, which may be the catheter 70, is advanced within the sEWC 14 and into the airways of the patient P's lungs in step 208. With the locatable endo-luminal device 70 disposed within the airways of the patient's lungs, in step 210, a plurality of locations of one or both of the EM sensors 14a, 72 is identified within the reference coordinate frame in an EM registration process as the catheter 70 is maneuvered within the airways of the patient P and stored within the memory 32. The position of the catheter 70 in the airways may be determined based on the EM registration process. In some aspects, the previously created pathway plan may be displayed to assist a clinician in maneuvering the catheter 70 through airways according to the pathway plan. In parallel with step 210, in step 212, real-time images are captured by the camera 74 of the catheter 70 as the catheter 70 is maneuvered within the airways of the patient P.

In step 214, the captured real-time images are segmented to identify anatomical landmarks within the captured real-time images. The captured real-time images may be segmented using a neural network model (e.g., a convolutional neural network model). In some aspects, airways and/or other features may be detected in the real-time images using the neural network model, which may be trained based on previous detection result data. Next, the next relevant airway may be determined according to the planned pathway to the target, the EM registration, and the detected airways and/or other features. Then, the next relevant airway may be presented to a user through a display to assist a clinician in navigating the catheter 70 to the target according to the planned pathway.

The anatomical landmarks identified within the real-time images are compared to the anatomical landmarks identified in the 3D representation of the airways of the patient P in step 216. In step 218, it is determined if there is a match between the anatomical landmarks identified in the real-time images and the anatomical landmarks identified in the 3D representation. If a match between the anatomical landmarks identified in the real-time images and the anatomical landmarks identified in the 3D representation is identified, in step 220, a distance between the camera 74 and the matched anatomical landmark within the real-time image is determined, and in step 222, the location of the catheter 70 within the reference coordinate frame is identified and stored in the memory 32. Optionally, if a match between the anatomical landmarks identified in the real-time images and the anatomical landmarks identified in the 3D representation is identified, in step 224, a plurality of virtual images is generated as being taken from a plurality of poses at the estimated location of the camera 74 of the catheter 70 within the 3D representation.

In step 226, the generated plurality of virtual images is compared to the captured real-time image and a virtual image having a similar view to the captured real-time image is identified. Using the identified virtual image, the location of the catheter 70 within the 3D representation is identified and stored in the memory 32 in step 222. If it is determined that there is no match in step 218, or the software application is unable to identify a match, between the anatomical landmarks identified in the real-time images and the anatomical landmarks identified in the 3D representation, the method returns to step 212. In embodiments, it is determined if all of the required portions of the airways of the patient P have been surveyed in step 228. If additional portions of the airways of the patient P need to be surveyed, the method returns to step 212. If it is determined that no further surveying is required, in step 230, the location data corresponding to the EM sensor 72 and the real-time images containing an anatomical landmark match is used to register the location of the catheter 70 to the 3D representation of the airways of the patient P and the method ends at step 232. As can be appreciated, the above-described method may be repeated as many times as necessary and may be performed both globally and locally within the airways of the patient P.

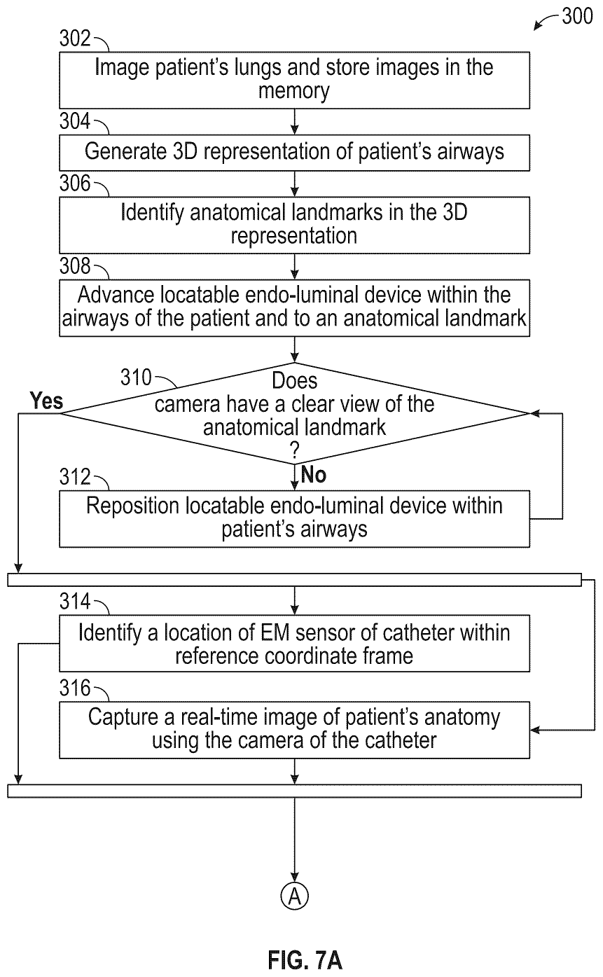

With reference to FIGS. 7A and 7B, another method of registering a location of a medical device to a 3D model of a patient's luminal network is described and generally identified by reference numeral 300. Initially, in step 302, the patient's lungs are imaged using any suitable imaging modality (e.g., for example, CT, MRI, or CBCT) and the images are stored on the memory 32 associated with the workstation 20. In step 304, the images stored on the memory 32 are utilized to generate and view a 3D representation of the airways of the patient P's lungs. Thereafter, anatomical landmarks, such as for example, a bifurcation, are identified within the 3D representation in step 306. Once the 3D representation of the airways is generated and anatomical landmarks are identified within the 3D representation, a locatable endo-luminal device, which may be the catheter 70, is advanced within the sEWC 14, into the airways of the patient P's lungs, and navigated to an anatomical landmark, such as the tracheal carina, within the patient's airways in step 308. In step 310, it is determined if the camera 74 of the catheter 70 has a clear view of the anatomical landmark. If it is determined that there is not a clear view of the anatomical landmark, the locatable endo-luminal device is repositioned in step 312 and the method returns to step 310. If it is determined that there is a clear view of the anatomical landmark, in step 314, a location of the EM sensor 14a and/or 72 is identified within the reference coordinate frame. In parallel with step 314, a real-time image of the patient's anatomy is captured by the camera 74 of the catheter 70 in step 316.

In step 318, the pose of the camera 74 of the catheter 70 relative to the 3D representation when the real-time image was captured is determined. Using the determined pose of the camera 74 and the identified location of the EM sensor 14a and/or 72, the location of the catheter 70 is registered to the 3D representation in step 320. In step 322, the catheter 70 is further advanced within the patient's airways, and in parallel with step 322, the location of the EM sensor 14a and/or 72 is periodically identified within the reference coordinate frame and saved in the memory 32 in step 324. In step 326, a point cloud is generated from the saved locations of the EM sensor 14a and/or 72 within the reference coordinate frame. In step 328, the point cloud is registered to the 3D representation and the registration of the catheter to the 3D representation is updated. In step 330, it is determined if the location of the catheter 70 within the patient's airways has changed (e.g., such as, further advanced or retracted within the patient's airways). If it is determined that the location of the catheter 70 has changed, the method returns to step 322. If it is determined that the location of the catheter 70 has not changed, the method ends at step 332. As can be appreciated, the above-described method may be repeated as many times as necessary and may be performed both globally and locally within the airways of the patient P.

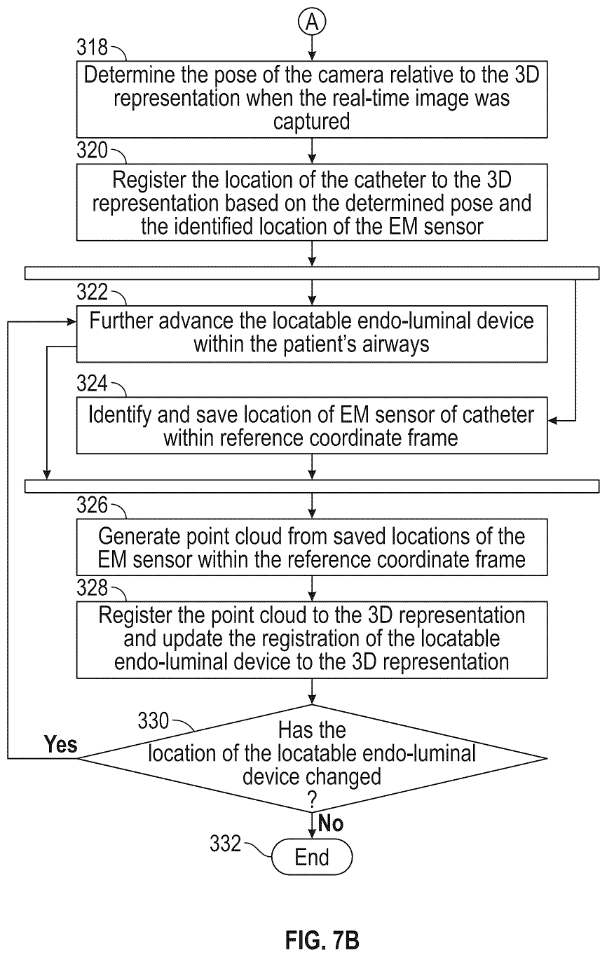

With reference to FIG. 8, another method of registering a location of a medical device to a 3D model of a patient's luminal network is described and generally identified by reference numeral 400. Initially, in step 402, the patient's lungs are imaged using any suitable imaging modality (e.g., for example, CT, MRI, or CBCT) and the images are stored on the memory 32 associated with the workstation 20. In step 404, the images stored on the memory 32 are utilized to generate and display a 3D representation of the airways of the patient P's lungs. Once the 3D representation of the airways is generated, a locatable endo-luminal device, which may be the catheter 70, is advanced within the sEWC 14 and into the airways of the patient P's lungs in step 406. In step 408, a location of the EM sensor 14a and/or 72 is identified within the reference coordinate frame according to an EM registration process. In parallel with step 408, a real-time image of the patient's anatomy is captured by the camera 74 of the catheter 70 in step 410.

In aspects, the EM registration may be updated according to the real-time image as follows. An initial catheter position in the 3D representation of the airways is determined according to the EM registration process. Next, airways and/or carinas may be detected in the real-time images using a neural network model, which may be trained based on previous detection result data. Then, the EM registration is updated based on the detected airways and/or carinas. For example, the catheter position and orientation in the 3D representation of the airways may be updated.

In step 412, a depth map is estimated from the captured real-time image, and in step 414, the estimated depth map is converted into a 3D point cloud. In step 416, the 3D point cloud is registered to the 3D representation and the method ends in step 418. As can be appreciated, the above-described method may be repeated as many times as necessary and may be performed both globally and locally within the airways of the patient P.

In aspects, a next airway may be presented (e.g., displayed) to a user according to a planned navigation pathway. This may include pre-procedurally creating a plan including a pathway to a target in the 3D representation. Then, during the procedure, the position of the catheter in the airways is determined according to the EM registration. Next, airways may be detected in the real-time images using a neural network model, which may be trained based on previous detection result data. Then, the next relevant airway on the pathway is determined according to the pathway to the target, the EM registration, and the detected airways. The next relevant airway may be presented to a user through a display. As described above with reference to FIGS. 6A and 6B, these features for presenting a next airway according to the planned navigation pathway may similarly be incorporated into the methods described above with reference to FIGS. 7A, 7B, and 8. For example, a neural network model may be used to detect landmarks or features within the real-time images (e.g., bronchoscope video).

In aspects, the EM registration may be updated according to the real-time images. After the 3D representation of the airway tree is created, initial catheter position in the airway tree is determined according to EM registration during the procedure. Next, airways and/or carinas may be detected in the real-time images using a neural network model, which may be trained based on previous detection result data. Then, the registration is updated based on the detected airways and/or carinas. For example, the catheter position and orientation in the 3D representation of the airway tree may be updated. As described above with reference to FIG. 8, these features for updating the EM registration based on the real-time images may similarly be incorporated into the methods described above with reference to FIGS. 6A, 6B, 7A, and 7B.

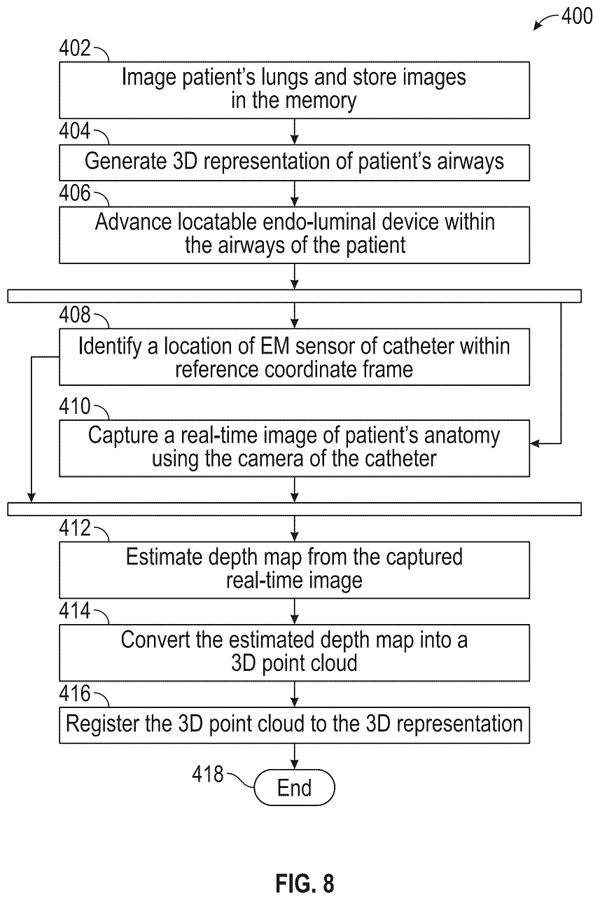

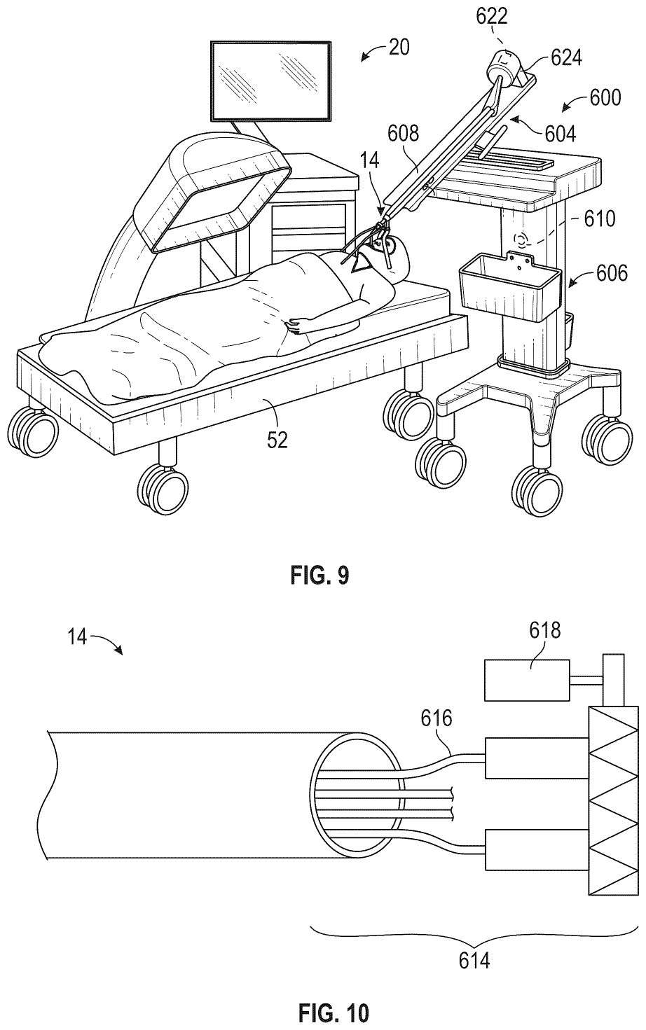

With reference to FIGS. 9 and 10, it is envisioned that the system 10 may include a robotic surgical system 600 having a drive mechanism 602 including a robotic arm 604 operably coupled to a base or cart 606, which may, in embodiments, be the workstation 20. The robotic arm 604 includes a cradle 608 that is configured to receive a portion of the sEWC 14. The sEWC 14 is coupled to the cradle 608 using any suitable means (e.g., for example, straps, mechanical fasteners, and/or couplings). It is envisioned that the robotic surgical system 600 may communicate with the sEWC 14 via electrical connection (e.g., for example, contacts and/or plugs) or may be in wireless communication with the sEWC 14 to control or otherwise effectuate movement of one or more motors (FIG. 10) disposed within the sEWC 14 and in embodiments, may receive images captured by a camera (not shown) associated with the sEWC 14. In this manner, it is contemplated that the robotic surgical system 600 may include a wireless communication system 610 operably coupled thereto such that the sEWC 14 may wirelessly communicate with the robotic surgical system 600 and/or the workstation 20 via Wi-Fi, Bluetooth®, for example. As can be appreciated, the robotic surgical system 600 may omit the electrical contacts altogether and may communicate with the sEWC 14 wirelessly or may utilize both electrical contacts and wireless communication. The wireless communication system 610 is substantially similar to the network interface 36 (FIG. 2) described hereinabove, and therefore, will not be described in detail herein in the interest of brevity. As indicated hereinabove, the robotic surgical system 600 and the workstation 20 may be one in the same, or in embodiments, may be widely distributed over multiple locations within the operating room. It is contemplated that the workstation 20 may be disposed in a separate location and the display 44 (FIGS. 1 and 2) may be an overhead monitor disposed within the operating room.

As indicated hereinabove, it is envisioned that the sEWC 14 may be manually actuated via cables or push wires, or for example, may be electronically operated via one or more buttons, joysticks, toggles, actuators (not shown) operably coupled to a drive mechanism 614 disposed within an interior portion of the sEWC 14 that is operably coupled to a proximal portion of the sEWC 14, although it is envisioned that the drive mechanism 614 may be operably coupled to any portion of the sEWC 14. The drive mechanism 614 effectuates manipulation or articulation of the distal end of the sEWC 14 in four degrees of freedom or two planes of articulation (e.g., for example, left, right, up, or down), which is controlled by two push-pull wires, although it is contemplated that the drive mechanism 614 may include any suitable number of wires to effectuate movement or articulation of the distal end of the sEWC 14 in greater or fewer degrees of freedom without departing from the scope of the present disclosure. It is contemplated that the distal end of the sEWC 14 may be manipulated in more than two planes of articulation, such as for example, in polar coordinates, or may maintain an angle of the distal end relative to the longitudinal axis of the sEWC 14 while altering the azimuth of the distal end of the sEWC 14 or vice versa. In one non-limiting embodiment, the system 10 may define a vector or trajectory of the distal end of the sEWC 14 in relation to the two planes of articulation.

It is envisioned that the drive mechanism 614 may be cable actuated using artificial tendons or pull wires 616 (e.g., for example, metallic, non-metallic, and/or composite) or may be a nitinol wire mechanism. In embodiments, the drive mechanism 614 may include motors 618 or other suitable devices capable of effectuating movement of the pull wires 616. In this manner, the motors 618 are disposed within the sEWC 14 such that rotation of an output shaft the motors 618 effectuates a corresponding articulation of the distal end of the sEWC 14.

Although generally described as having the motors 618 disposed within the sEWC 14, it is contemplated that the sEWC 14 may not include motors 618 disposed therein. Rather, the drive mechanism 614 disposed within the sEWC 14 may interface with motors 622 disposed within the cradle 608 of the robotic surgical system 600. In embodiments, the sEWC 14 may include a motor or motors 618 for controlling articulation of the distal end 14b of the sEWC 14 in one plane (e.g., for example, left/null or right/null) and the drive mechanism 624 of the robotic surgical system 600 may include at least one motor 622 to effectuate the second axis of rotation and for axial motion. In this manner, the motor 618 of the sEWC 14 and the motors 622 of the robotic surgical system 600 cooperate to effectuate four-way articulation of the distal end of the sEWC 14 and effectuate rotation of the sEWC 14. As can be appreciated, by removing the motors 618 from the sEWC 14, the sEWC 14 becomes increasingly cheaper to manufacture and may be a disposable unit. In embodiments, the sEWC 14 may be integrated into the robotic surgical system 600 (e.g., for example, one piece) and may not be a separate component.

From the foregoing and with reference to the various figures, those skilled in the art will appreciate that certain modifications can be made to the disclosure without departing from the scope of the disclosure.

Although the description of computer-readable media contained herein refers to solid-state storage, it should be appreciated by those skilled in the art that computer-readable storage media can be any available media that can be accessed by the processor 30. That is, computer readable storage media may include non-transitory, volatile, and non-volatile, removable and non-removable media implemented in any method or technology for storage of information such as for example, computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by the workstation 20.

The invention may be further described by reference to the following numbered paragraphs:

1. A surgical system, comprising:
a catheter including a camera and an electromagnetic (EM) sensor; and
a workstation operably coupled to the catheter, the workstation including processing means configured to:
   receive pre-procedure images of a patient's anatomy;
   generate a 3-dimensional (3D) representation of the patient's anatomy based on the received pre-procedure images;
   identify first anatomical landmarks within the generated 3D representation of the patient's anatomy;
   identify a location of the EM sensor of the catheter within a reference coordinate frame using the EM sensor;
   receive real-time images of the patient's anatomy from the camera of the catheter;
   identify second anatomical landmarks within the received real-time images corresponding to the identified first anatomical landmarks within the generated 3D representation of the patient's anatomy;
   identify a location of the camera within the reference coordinate frame using the identified second anatomical landmarks within the real-time images corresponding to the identified first anatomical landmarks within the generated 3D representation of the patient's anatomy; and
   register a location of the catheter to the 3D representation of the patient's anatomy using the identified locations of the EM sensor and the camera within the reference coordinate frame.

2. The system according to paragraph 1, wherein the processing means is configured to determine a distance between the camera and an identified anatomical landmark of the identified second anatomical landmarks within the received real-time images.

3. The system according to paragraph 2, wherein the EM sensor of the catheter is disposed on the catheter at a predetermined distance from the camera.

4. The system according to paragraph 3, wherein the processing means is configured to determine a distance between the camera and the identified anatomical landmark of the identified second anatomical landmarks within the received real-time images using the predetermined distance between the EM sensor and the camera.

5. The system according to paragraph 1, wherein the processing means is further configured to:
generate a plan including a pathway to a target in the 3D representation;
determine a position of the catheter in airways according to an EM registration based on the location of the EM sensor;
detect the airways in the real-time images using a neural network model;
determine a next airway on the pathway based on the pathway to the target, the EM registration, and the detected airways; and
display a representation of the next airway.

6. The system according to paragraph 1, wherein the processing means is configured to continuously receive real-time images of the patient's anatomy captured by the camera as the catheter is navigated through a luminal network of the patient.

7. The system according to paragraph 6, wherein the processing means is configured to continuously identify second anatomical landmarks within the received real-time images corresponding to the identified first anatomical landmarks within the generated 3D representation of the patient's anatomy as the catheter is navigated through the luminal network of the patient.

8. A surgical system, comprising:
a catheter having a camera configured to capture images of a patient's anatomy;
an extended working channel (EWC), the EWC configured to selectively receive the catheter and permit the catheter to access a luminal network of the patient, wherein the EWC includes an electromagnetic (EM) sensor; and
a workstation operably coupled to the catheter, the workstation including processing means configured to:
   generate a 3-dimensional (3D) representation of the patient's anatomy based on pre-procedure images of the patient's anatomy;
   identify first anatomical landmarks within the generated 3D representation of the patient's anatomy;
   receive real-time images of the patient's anatomy from the camera of the catheter;
   identify second anatomical landmarks within the received real-time images corresponding to the identified first anatomical landmarks within the generated 3D representation of the patient's anatomy;
   identify a location of the catheter within the reference coordinate frame using the identified second anatomical landmarks within the real-time images corresponding to the identified first anatomical landmarks within the generated 3D representation of the patient's anatomy; and
   register a location of the catheter to the 3D representation of the patient's anatomy using the identified location of the catheter within the reference coordinate frame.

9. The surgical system according to paragraph 8, wherein the processing means is configured to identify a location of the EM sensor of the EWC within the reference coordinate frame, wherein the location of the catheter is registered to the 3D representation of the patient's anatomy using both the identified location of the EM sensor and the identified location of the catheter; or wherein the camera is disposed a predetermined distance beyond the EM sensor of the EWC.

10. The surgical system according to paragraph 9, wherein the catheter is configured to transition between a first, locked position where the catheter is inhibited from moving relative to the EWC and a second, unlocked position where the catheter is permitted to move relative to the EWC; or
wherein the processing means is configured to determine a distance between the camera and an identified anatomical landmark of the identified second anatomical landmarks within the received real-time images using the predetermined distance between the EM sensor and the camera.

11. The surgical system according to paragraph 8, wherein the processing means is configured to continuously receive real-time images of the patient's anatomy captured by the camera as the catheter is navigated through a luminal network of the patient; and
wherein the processing means is configured to continuously identify second anatomical landmarks within the received real-time images corresponding to the identified first anatomical landmarks within the generated 3D representation of the patient's anatomy as the catheter is navigated through the luminal network of the patient.

12. A method of operating a surgical system, the method comprising:
generating a 3-dimensional (3D) representation of a patient's luminal network based on pre-procedure images of the patient's anatomy;
identifying first anatomical landmarks within the generated 3D representation of the patient's luminal network;
identifying a plurality of locations of an electromagnetic (EM) sensor disposed on a catheter as the catheter is navigated through the luminal network of the patient;
receiving a plurality of real-time images captured by a camera disposed on the catheter as the catheter is navigated through the luminal network of the patient;
identifying second anatomical landmarks within the received real-time images corresponding to the identified first anatomical landmarks within the generated 3D representation of the patient's anatomy;
identifying a location of the camera within the reference coordinate frame using the identified second anatomical landmarks within the real-time images corresponding to the identified first anatomical landmarks within the generated 3D representation of the patient's anatomy; and
registering a location of the catheter to the 3D representation of the patient's anatomy using the identified positions of the EM sensor and the camera within the reference coordinate frame.

13. The method according to paragraph 12, further comprising determining a distance between the camera and an identified anatomical landmark of the identified second anatomical landmarks within the received real-time images, wherein the location of the camera within the reference coordinate frame is identified using a pre-determined distance between the EM sensor and the camera,
wherein the distance between the camera and the identified anatomical landmark of the identified second anatomical landmarks within the received real-time images is determined using a pre-determined distance between the EM sensor and the camera.

14. The method according to paragraph 12, further comprising:
determining initial catheter position in the 3D representation based on registering a location of the catheter to the 3D representation;
identifying an airway and/or a carina in the received real-time images using a neural network model; and
updating registering the location of the catheter to the 3D representation based on the identified airway and/or carina.

15. The method according to paragraph 14, wherein identifying second anatomical landmarks within the received real-time images includes continuously analyzing the continuously received plurality of real-time images to identify second anatomical landmarks within the received real-time images corresponding to the identified first anatomical landmarks within the generated 3D representation of the patient's anatomy.

What is claimed is:

1. A system for performing a surgical procedure, comprising:
a catheter including a camera and an electromagnetic (EM) sensor; and
a workstation operably coupled to the catheter, the workstation including a memory and a processor, the memory storing instructions, which when executed by the processor cause the processor to:
receive pre-procedure images of a patient's anatomy;
generate a 3-dimensional (3D) representation of the patient's anatomy based on the received pre-procedure images;
identify first anatomical landmarks within the generated 3D representation of the patient's anatomy;
identify a location of the EM sensor of the catheter within a reference coordinate frame using the EM sensor;
receive real-time images of the patient's anatomy from the camera of the catheter;
identify second anatomical landmarks within the received real-time images corresponding to the identified first anatomical landmarks within the generated 3D representation of the patient's anatomy;
identify a location of the camera within the reference coordinate frame using the identified second anatomical landmarks within the real-time images corresponding to the identified first anatomical landmarks within the generated 3D representation of the patient's anatomy; and
register a location of the catheter to the 3D representation of the patient's anatomy using the identified locations of the EM sensor and the camera within the reference coordinate frame.

2. The system according to claim 1, further comprising the memory storing thereon further instructions, which when executed by the processor cause the processor to determine a distance between the camera and an identified anatomical landmark of the identified second anatomical landmarks within the received real-time images.

3. The system according to claim 2, wherein the EM sensor of the catheter is disposed on the catheter at a predetermined distance from the camera.

4. The system according to claim 3, further comprising the memory storing thereon further instructions, which when executed by the processor cause the processor to determine a distance between the camera and the identified anatomical landmark of the identified second anatomical landmarks within the received real-time images using the predetermined distance between the EM sensor and the camera.

5. The system according to claim 1, further comprising an extended working channel (EWC), the EWC configured to selectively receive the catheter and permit the catheter to access a luminal network of the patient.

6. The system according to claim 1, further comprising the memory storing thereon further instructions, which when executed by the processor cause the processor to continuously receive real-time images of the patient's anatomy captured by the camera as the catheter is navigated through a luminal network of the patient.

7. The system according to claim 6, further comprising the memory storing thereon further instructions, which when executed by the processor cause the processor to continuously identify second anatomical landmarks within the received real-time images corresponding to the identified first anatomical landmarks within the generated 3D representation of the patient's anatomy as the catheter is navigated through the luminal network of the patient.

8. A system for performing a surgical procedure, comprising:
a catheter having a camera configured to capture images of a patient's anatomy;
an extended working channel (EWC), the EWC configured to selectively receive the catheter and permit the catheter to access a luminal network of the patient, wherein the EWC includes an electromagnetic (EM) sensor; and
a workstation operably coupled to the catheter, the workstation including a memory and a processor, the memory storing instructions, which when executed by the processor cause the processor to:
generate a 3-dimensional (3D) representation of the patient's anatomy based on pre-procedure images of the patient's anatomy;
identify first anatomical landmarks within the generated 3D representation of the patient's anatomy;
receive real-time images of the patient's anatomy from the camera of the catheter;
identify second anatomical landmarks within the received real-time images corresponding to the identified first anatomical landmarks within the generated 3D representation of the patient's anatomy;
identify a location of the catheter within the reference coordinate frame using the identified second anatomical landmarks within the real-time images corresponding to the identified first anatomical landmarks within the generated 3D representation of the patient's anatomy; and
register a location of the catheter to the 3D representation of the patient's anatomy using the identified location of the catheter within the reference coordinate frame.

9. The system according to claim 8, further comprising the memory storing thereon further instructions, which when executed by the processor cause the processor to identify a location of the EM sensor of the EWC within the reference coordinate frame, wherein the location of the catheter is registered to the 3D representation of the patient's anatomy using both the identified location of the EM sensor and the identified location of the catheter.

10. The system according to claim 8, wherein the camera is disposed a predetermined distance beyond the EM sensor of the EWC.

11. The system according to claim 9, wherein the catheter is configured to transition between a first, locked position where the catheter is inhibited from moving relative to the EWC and a second, unlocked position where the catheter is permitted to move relative to the EWC.

12. The system according to claim 10, further comprising the memory storing thereon further instructions, which when executed by the processor cause the processor to determine a distance between the camera and an identified anatomical landmark of the identified second anatomical landmarks within the received real-time images using the predetermined distance between the EM sensor and the camera.

13. The system according to claim 8, further comprising the memory storing thereon further instructions, which when executed by the processor cause the processor to continuously receive real-time images of the patient's anatomy captured by the camera as the catheter is navigated through a luminal network of the patient.

14. The system according to claim 13, further comprising the memory storing thereon further instructions, which when executed by the processor cause the processor to continuously identify second anatomical landmarks within the received real-time images corresponding to the identified first anatomical landmarks within the generated 3D representation of the patient's anatomy as the catheter is navigated through the luminal network of the patient.

15. A method of registering a location of a medical device to a 3D representation of a patient's luminal network, comprising:
generating a 3-dimensional (3D) representation of a patient's luminal network based on pre-procedure images of the patient's anatomy;
identifying first anatomical landmarks within the generated 3D representation of the patient's luminal network;
identifying a plurality of locations of an electromagnetic (EM) sensor disposed on a catheter as the catheter is navigated through the luminal network of the patient;
receiving a plurality of real-time images captured by a camera disposed on the catheter as the catheter is navigated through the luminal network of the patient;
identifying second anatomical landmarks within the received real-time images corresponding to the identified first anatomical landmarks within the generated 3D representation of the patient's anatomy;
identifying a location of the camera within the reference coordinate frame using the identified second anatomical landmarks within the real-time images corresponding to the identified first anatomical landmarks within the generated 3D representation of the patient's anatomy; and
registering a location of the catheter to the 3D representation of the patient's anatomy using the identified positions of the EM sensor and the camera within the reference coordinate frame.

16. The method according to claim 15, further comprising determining a distance between the camera and an identified anatomical landmark of the identified second anatomical landmarks within the received real-time images, wherein the location of the camera within the reference coordinate frame is identified using the determined distance.

17. The method according to claim 16, wherein the distance between the camera and the identified anatomical landmark of the identified second anatomical landmarks within the received real-time images is determined using a pre-determined distance between the EM sensor and the camera.

18. The method according to claim 15, further comprising advancing the catheter within an extended working channel (EWC) to gain access to the patient's luminal network.

19. The method according to claim 15, wherein receiving a plurality of real-time images captured by the camera includes continuously receiving the plurality of real-time images from the camera as the catheter is navigated through the luminal network of the patient.

20. The method according to claim 19, wherein identifying second anatomical landmarks within the received real-time images includes continuously analyzing the continuously received plurality of real-time images to identify second anatomical landmarks with the received real-time images corresponding to the identified first anatomical landmarks within the generated 3D representation of the patient's anatomy.
